(19) United States
(12) Patent Application Publication
(10) Pub. No.: US 2005/0042604 A1
(43) Pub. Date: Feb. 24, 2005

(54) Novel nucleic acid-based tracers and their uses thereof

(75) Inventors: Sun-Wing Tong, Hong Kong (HK); Chun Liang, Hong Kong (HK)

Correspondence Address: Kent H. Cheng, Esq.;Cohen, Pontani, Lieberman & Pavane, Suite 1210, 551 Fifth Avenue, New York, NY, 10176, US

(73) Assignee: Grandtech Limited

(21) Appl. No.: 10/909,445

(22) Filed: Aug. 2, 2004

Related U.S. Application Data
(60) Provisional application No. 60/493,182, filed on Aug. 7, 2003.
(60) Provisional application No. 60/510,207, filed on Oct. 10, 2003.

(51) Int. Cl. C12Q 1/70; G01N 33/554; G01N 33/569

(52) U.S. Cl. 435/5; 435/7.32

(57) ABSTRACT

The present invention relates to a method of determining whether a facility can effectively prevent the spread of an infectious disease by a) releasing an effective amount of biological tracers in or near the facility, to mimic the spread of pathogens that cause the infectious disease; and b) detecting the presence or amount of the biological tracers to determine the spread of the biological tracers in the environment of the facility. The present invention further provides a method and apparatus to generate aerosol that mimic the human sneeze and cough so as to more realistically determine the spread of an infectious disease by such a source.

### RELATED APPLICATIONS

This application claims priority from U.S. Provisional Patent Applications Ser. Nos. 60/493,182 which was filed on Aug. 7, 2003; 60/510,207 which was filed on Oct. 10, 2003.

### BACKGROUND OF THE INVENTION

1. Field of the Invention

To prevent cross-infection in hospitals through aerosol generated by patients, medical intervention or other activities, each ward and even each bed should have separate ventilation from the others. This invention provides methods to determine if such conditions are met. In addition, this invention provides novel tools to experimentally model and determine the extent of spread of an outbreak of respiratory infectious disease, and the tracing of pollution source for surface and underground water and the atmosphere.

The current invention further provides portable lightweight apparatuses and methods of generating aerosol, carrying mimics of viruses and bacteria to simulate the production of infectious aerosols by human beings during activities that result in forceful ejection of lung air, such as coughing and sneezing.

This inventions also provides methods of collecting a sample of ambient air for analysis of its constituents, principally aerosolized macromolecules for the simulation of spread of air-borne pathogens and hence the evaluation of the adequacy of ventilation and the safety from air-borne pathogens in hospital wards and laboratories.

Other constituents in the air that may be sampled are rare isotopes of air, biological substances (pollen, viruses, bacteria, fungal spores), and chemicals not normally present but released because of natural phenomena (e.g. methane, sulfur oxides, carbon monoxide, carbon dioxide) or human activities (benign intentional, malicious intentional, investigational intentional, unintentional disastrous or unintentional investigational, e.g. the investigational release of a tracer gas, or other types of tracer molecules for the study or certification of ventilation work or the modeling of disease spread).

2. Background

With the tremendous improvements to human health brought about by the provision of clean water, the air we breathe becomes the last frontier. Indoors air pollution and worse, cross infection by respiratory pathogens are now serious threats to the health of gregarious human beings. Highly contagious air-borne diseases that spread from person to person have the potential to exponentially infect large numbers of people and cause considerable strains to the health care system, the well-being of the public, the economy and beyond.

Airport and train terminals, the subway (underground) systems, hotels, convention and exhibition centers, museums and public libraries, government offices and schools are some examples of places where large numbers of people congregate and come and go. Hospitals are the physical solutions to managing complicated diseases in our ageing populations that require multidisciplinary teamwork and expensive, often gigantic equipment.

Most hospitals have hundreds of patients and workers. Yet, most are outdated when confronted by such threats as air-borne pathogens and in-door air pollution. Hospital wards continue to accommodate more than one patient in the same room and are still lacking independent ventilation from bed to bed. The result is that air-borne infectious diseases have a chance to spread from one patient to other patients before the crisis is identified.

Infectious disease hospitals or wards are the psychological gatekeepers in an outbreak of air-borne infectious disease. If occupants in those facilities are safe from nosocomial infection, confidence in winning the war against the pathogen can be upheld. On the other hand, if health care workers fall in droves, then the health care system, and eventually the public will degenerate into panic.

To ensure that the facilities are safe from such events, future buildings need to be rigorously designed and tested. Current buildings also need to be tested for safety against such events. Any modifications for improvements in ventilation and drainage systems must be further tested to ensure that such work meet the intended standards. A reliable means of testing needs to be in place.

3. Prior Art

Tracers

Previous methods of testing ventilation relied on smoke or gaseous tracers and technologies for their detection, ranging from smoke sensors, to chemical sensors and laser imaging. Whereas the sensitivity can be quite high, especially in regard to detection by the latest gas chromatographic instruments, the degree of sensitivity may not meet the more stringent requirements of an infectious disease hospital. However, more often than not, no testing is performed on the assumption that the provision is adequate.

There are over 75 gaseous tracers (http://www.gasimaging.com/GasesDetected.html). The two commonly used gaseous tracers, SF6 and PFT, have sensitivities in the range of one in a trillion (10⁻¹²) and quadrillion (10⁻¹⁵), respectively.

This range of sensitivity is far less than that of nucleic acid-based tracers, single copies of which are detectable.

Aerosol Generators and Air Samplers

In modeling and investigating the spread of air-borne diseases, such as tuberculosis and viral respiratory illnesses, or in studying the hospital ward environment for safety from such air-borne diseases, the ability to generate aerosol in the size range produced by humans, and in conditions as similar as possible to sneezing and coughing, is very important.

The importance lies in the fact that only by simulating the human can we get an accurate assessment of risk and formulate an achievable goal for the improvement of the hospital environment.

Some methods of generating aerosol produce a skewed spectrum of aerosol with size of aerosol droplets that is dissimilar to those produced by humans during coughing and sneezing. Aerosol droplets that are too large do not mimic small aerosols that preferentially deposit in the lungs rather than the upper airways. On the other hand, aerosol droplets that are uniformly very small, when coupled with some degree of airflow, may give a false negative result in locations where larger droplets might settle because of the direction of ejection and the speed of the aerosol.

These aerosol generators do not normally generate pulses or are difficult to configure to generate pulses that mimic human sneezing and coughing. Rather, a continuous stream of aerosol is produced. In addition to not simulating the human, these aerosol generators employ volatile tracer gases, giving results that are impossible to interpret. Release of such tracer gases in a confined space does not simulate virus spread by aerosol. This is because gas molecules rapidly take up the entire space of the container by diffusion; any tracer gas introduced in the hospital ward quickly spreads throughout the ward and beyond, making it impossible to mimic the spread of infectious disease agents by aerosol droplets, which has a different distribution dynamics by virtue of the greater effects of gravity on the droplets.

Many existing aerosol generators are also bulky and require excessive solution to prime the machine, making them difficult to use in certain circumstances and with certain reagent solutions.

Portable aerosol generators of the kind that produce low-pressure aerosol and larger droplets are available, primarily for the delivery of medications to the bronchial mucosal for the treatment of asthma (U.S. Pat. Nos. 4,566,452; 5,007,419; 5,040,527; 6,567,686; 6,615,824). The low ejection velocity and the larger droplets may not fully simulate a cough or sneeze.

Without an effective simulation of human sneezing and coughing by a suitably powerful portable aerosol generator that generates a satisfactory range of aerosol droplets and appropriate ejection velocity, it is impossible to assess the real risk of nosocomial air-borne communicable diseases in a hospital ward environment.

After the release of aerosol, the ability to quickly sample a representative sample of air at the site being studied or investigated is an important arm in the process of studying aerosol distribution. Being able to achieve this rapidly is important because multiple samples need to be taken at multiple sites over the period of investigation. Such samples also provide snapshots of the situation at the time the samples are taken. Multiple such snapshots can be stitched together to reconstruct the temporal characteristics of the aerosol distribution.

Various methods of air sampling can be used, such as pumping air through a liquid medium (U.S. Pat. Nos. 6,605,446; 6,619,143; 6,477,906; 6,550,347), passing air close to the surface of a medium (U.S. Pat. Nos. 6,446,514; 6,565,638), impacting air against the surface of a medium (U.S. Pat. Nos. 6,514,721; 6,463,814), by activating aerosol and its collection into a liquid stream (U.S. Pat. No. 6,506,345) or by splitting an air current and the deceleration and analysis of one representative current (U.S. Pat. No. 6,553,848). By collecting a representative sample of the constituent of interest, microbiological culture or chemical analysis is then carried out.

All of these methods are either qualitative or requiring extended time for sampling, thereby failing to give quantitative data or to provide an accurate snapshot of the status at a given point in time. Failing to provide an accurate snapshot also means inability to take multiple snapshots to re-construct the sequence of events in the dynamic evaluation of airflow.

### SUMMARY OF THE INVENTION

This invention provides custom-designed synthetic oligonucleotides, natural DNA molecules, and derivatives thereof as tracers and their detection by nucleic acid amplification and other means. The sensitivity of detection of the tracer is limited only by the ability to sample it (recover from the environment) and not by its concentration or absolute quantity.

The present invention also provides portable apparatuses and methods for the generation of aerosol droplets in the range produced by human beings during coughing and sneezing, to enable the release of tracer molecules attached to such aerosol droplets and its subsequent detection, so as to determine the relative safety of an environment against the spread of air-borne respiratory pathogens.

In addition, this invention provides methods for rapidly acquiring representative samples of air at a site of interest, and for the isolation, transportation, and extraction of an air-borne substance of interest, so that subsequent laboratory detection and quantification can be carried out.

This invention also provides a means of reconstructing the dynamics of airflow through multiple samples taken over a period of time, as well as providing a means to study the spatial distribution of aerosol droplets at a given place under different ventilation arrangements and human activities.

Definitions

“Oligonucleotide” means DNA, RNA, single-stranded or double-stranded and any chemical modifications thereof. Modifications include, but are not limited to those, which change or provide other chemical groups.

“Tracer” means a substance composed of many identical items, usually molecules, that is dispersible and when subsequently recovered and positively identified, provide information that can be inferred from its presence.

“Nosocomial infection” means the spread of an infectious disease from patient(s) within a hospital or health care facility to other patients.

“Hybridization” used in this document means fusion of two single complementary DNA strands (DNA/DNA hybridization), or the fusion of complementary DNA and RNA strands (DNA/RNA hybridization).

“Restriction site” means a short DNA sequence, often 4-8 base pairs long and usually palindromic, which is recognized by a restriction endonuclease.

“Polymorphism” means the existence of two or more variants (alleles, phenotypes, sequence variants, chromosomal structure variants) at significant frequencies in the population.

“Restriction fragment length polymorphism” means a polymorphic difference in the size of allelic restriction fragments as a result of the polymorphic presence or absence of a particular restriction site.

“Polymerase chain reaction (PCR)” refers to a technique for making many copies of a stretch of DNA sequence in the test tube. It employs repetitive thermal cycling consisting of denaturation of double-stranded DNA, annealing of appropriate oligonucleotide primers, and extension of the primer by polymerase enzyme.

“GenBank”—GenBank® is the NIH genetic sequence database, an annotated collection of all publicly available DNA sequences.

### BRIEF DESCRIPTION OF THE DRAWINGS

FIG. 1 depicts a six-bed cubicle in a hospital ward.

FIG. 2 shows the preparation of one of the beds for the recovery of tracer.

FIG. 3 depicts the release of tracer as aerosols in another bed as a simulation of the source of an air-borne infectious disease.

FIG. 4 is a schematic of the manually operated air sampling apparatus whereby environmental air is sampled by pulling a piston.

FIG. 5 depicts another embodiment of an air sampling apparatus where the rubber-stoppered vacuum tube draws in air when punctured momemtarily by a hollow needle.

### DETAILED DESCRIPTION OF DRAWINGS

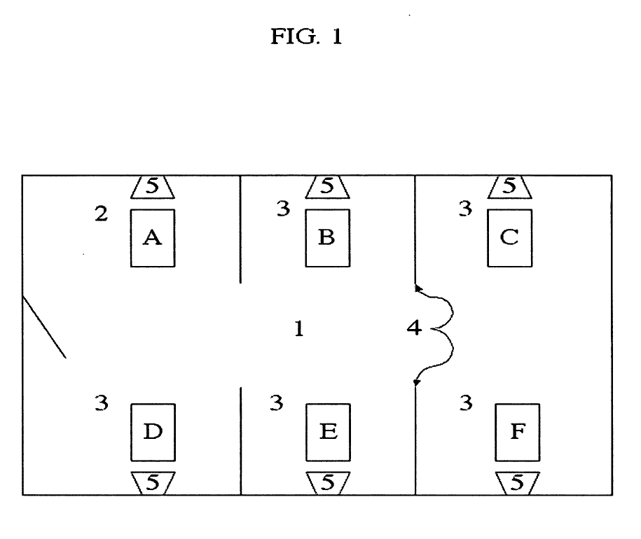

FIGS. 1-3 are described in detail in EXAMPLE 2 (see below).

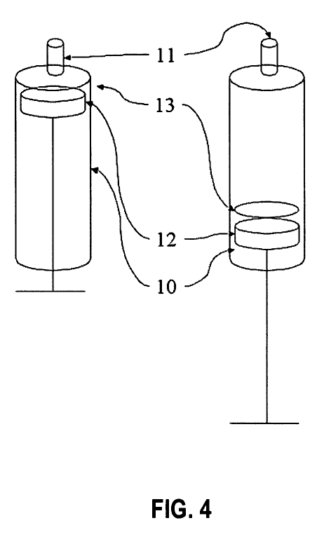

FIG. 4 shows the essential components of one embodiment of an air sampling apparatus. The container (10) is sealed at the intake port (11) before use. A piston (12) travels along the inside of the container, which is airtight. Pulling the piston draws air into the container when the seal at the intake port (11) is removed. In the container is a predetermined quantity of DNase-free buffered electrolyte solution (13). After air is sampled, the intake port (11) is again sealed until brought back to the laboratory for analysis.

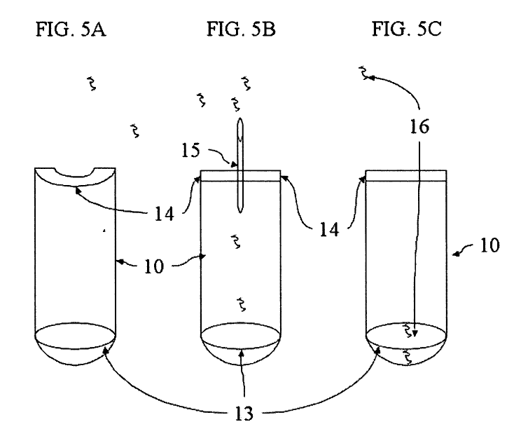

FIG. 5A depicts another embodiment of the air sampler, comprising a container (10) with only one open end, sealed by a rubber stopper (14). The rubber stopper (14) is schematically curved to illustrate the vacuum inside the container. The apparatus contains a predetermined volume of DNase-free buffered electrolyte solution (13).

FIG. 5B shows air sample being collected by puncturing the rubber stopper (14) by a hollow needle (15), in this case, admitting tracer molecules (16) present in the air being sampled.

FIG. 5C. After removal of the hollow needle, air inside the container is cut-off from outside air. The tracer molecule (16) goes into solution and is ready to be extracted in the laboratory for analysis.

### DETAILED DESCRIPTION OF THE PREFERRED EMBODIMENTS

Tracer

This invention employs the novel use of synthetic oligonucleotides, natural DNA molecules, and derivatives thereof as tracers (Examples 1-3). These molecules carry sequence information that can be made unique by special design and comparison against a database (GenBank) of naturally occurring DNA in catalogued living things. Each oligonucleotide can be of different length, but preferably between 30 and 150, more preferably between 50 and 120, and even more preferably between 60 and 80 nucleotides. Synthetic oligonucleotides can also be linked together to form longer tracers. Natural DNA molecules can be of various lengths, preferably between 100 and 3,000 nucleotides.

Design of Oligonucleotide Tracer

Design of these oligonucleotides can be achieved by:
1. randomly generating a string of A, T, G and C by tapping the keyboard
2. randomly altering a naturally occurring sequence of DNA or
3. removing the spaces and punctuations in an English language document and extracting all the As, Ts, Gs, and Cs to constitute the sequence of the oligonucleotide
4. Any other ways of generating random oligonucleotide sequences.

The oligonucleotides can be used without chemical modification. Alternatively, they can be chemically modified by adding other moieties during synthesis, changing to other forms such as phosphorothioates, or using a sugar other than deoxyribose or ribose in the backbone. The modification must not make the molecule unstable or unreadable by the polymerase enzyme in the amplification step.

The oligonucleotides can be single or double stranded DNA or RNA, DNA/RNA hybrids, or chimeric DNA/RNA molecules. The DNA oligonucleotides (oligodeoxyribonucleotides) can be made into double stranded and then inserted in a vector or plasmid, which can be propagated in bacterial, yeast or other cells. The DNA oligonucleotides can also be incorporated into the genome of an organism such as E. coli, yeast, or other cells, and total DNA from such an organism can be extracted and used as tracers.

The oligonucleotides and derivatives thereof may be used as naked DNA or RNA but preferably are encapsulated within liposomes, virus-like self-assembly of Human papillomavirus L1 protein or other proteins, or other self-assembled biological molecules or polymers that have a relatively neutral electrical charge. This will make the oligonucleotide stick less avidly to objects, more stable, better simulate viral and other pathogens, and easier to recover from the environment for analysis.

How to Make Sure that Oligonucleotide Tracer is Unique

To ensure that the sequence is unique, that is not normally encountered in the environment, two methods are used.

1. A query is performed by accessing several tools at the NCBI BLAST Homepage (http://www.ncbi.nim.nih.gov/BLAST/). The tools used to query the database include Standard nucleotide-nucleotide BLAST, MEGABLAST and “Search for short nearly exact matches”. By entering the sequence information into a database query (BLAST), it is possible to align the sequence against any known sequence in the biosphere and modifying it to make it unique. Virtually unlimited number of unique sequences can be designed because of the virtually unlimited combinations and permutations of the 4 nucleotides (adenine, thymine (or uracil, in RNA), guanine and cytosine) that make up the oligonucleotides, given the virtually unlimited lengths of the oligonucleotide sequences. The number of possible combinations in relation to the length of the oligonucleotide is expressed by the equation: C=4ⁿ, where C is number of combinations and n is the number of nucleotides making up the oligonucleotide.

The oligonucleotide tracer that shows no significant matches with any known sequences are entered into a new database kept by the inventors. They will be compared against each other at a later date, when necessary.

2. To make sure that the oligonucleotide is not present in the environment, an optional step is to perform a field test. To do so, a method of detection is prepared. One method to detect the presence of the oligonucleotide is to use the technique of polymerase chain reaction (PCR). A pair of primers is designed to amplify a region of the oligonucleotide. Material is collected from the environment, brought back to the laboratory and tested with these primers. The primers are also tested against the oligonucleotide. If reaction products are detected in the presence of the oligonucleotide but not in the environmental samples before dispersion of the tracer, then the oligonucleotide is indeed unique and can be used as a tracer.

Information about the Oligonucleotide can be Encoded

Information about the tracer, such as manufacturer, date of manufacture, sequence identification and lot number can be encoded within or outside the region amplified by the primers. For example, using a seven-bit sequence of only two nucleotides, such as adenine and thymine to represent 0 and 1, respectively, and using the American Standard Code for Information Interchange (ASCII), the following sequence would designate the number 8: ATTTAAA (0111000). The capital letter T would be represented by the sequence: TATATAA (1010100). The start of this digital barcode can be a run of 4 G's, or other sequence combinations, whereas the stop is designated by another sequence. Alternatively, using a code of 10 nucleotides to represent an English word, 1,048,576 (4¹⁰) different words can be encoded. Without using a very long sentence; a single English word, such as the name “Richard” can be designated to mean oligonucleotide manufactured by XXX company on Sep. 1, 2003 with sequence ID of 238888, lot number 238.

Natural Nucleic Acids as Tracers

DNA or RNA molecules consisting of natural sequences from any organisms can also be used as tracers. These nucleic acids can be generated by PCR, restriction digestion, transcription, chemical synthesis, etc. Before natural nucleic acids are used as tracers, it can be determined that these molecules are not present in the test environment using the methods described above. In addition, DNA or RNA isolated from organisms may also be used as tracers, as long as the test site is devoid of such nucleic acids.

Using the Tracer as an Environmental Investigation Tool

To be used as a tracer, the oligonucleotide, natural nucleic acid or their encapsulated form is dissolved or suspended in an environmentally friendly liquid such as water or buffers. The oligonucleotides, natural DNA, and derivatives can also be dissolved in a solution of gelatin, glycerol, or other substance that makes the solution viscous in order to more closely mimic viruses present in body fluids as they are released into the environment.

A typical stock solution of oligonucleotide or natural nucleic acid tracers can have a concentration of from 1 to 1,000 mmole/L but preferably from 10 to 100 mmole/L. This stock solution is usually diluted 10 to 10⁶ fold before use.

Other ingredients present include stabilizers to keep the liposomes or virus-like particles from aggregating, preservatives to prevent the growth of bacteria, algae or fungi, and buffer salts to maintain a predetermined pH.

Of note is the lack of DNase, RNase, lipase and proteinase in the solution by the careful preparation of the mixture in vessels and water free of those contaminants.

Before use, the stock solution is diluted in the laboratory, taking special precautions as outlined above. Oligonucleotides and natural nucleic acids are handled in special hoods and rooms in order to prevent the contamination of the laboratory, the different stock bottles and personnel or the inadvertent discharge of the oligonucleotide or natural nucleic acid tracer into the exterior.

In addition, a sample is taken before actual testing to determine the presence of and the concentration of the oligonucleotide or natural nucleic acid tracer.

As an added precaution, a portion of the tracer solution is kept in the vessel and brought back to the laboratory after field studies to determine if the oligonucleotide or natural nucleic acid tracer had remain stable and still detectable throughout field test.

For ventilation studies, the oligonucleotide or natural nucleic acid tracer is released into the atmosphere at the place of study, such as a hospital ward. To do this, the oligonucleotide or natural nucleic acid, which is in solution, need to be suspended in air.

One way to achieve this is to use a machine that makes uniformly sized droplets that measure from 1 to 5 micrometers in diameter, depending on whether airborne disease or droplet-disseminated disease is simulated. This is because airborne diseases are generally caused by small nuclei that are equal to or less than 1 micrometer in diameter, whereas droplet-disseminated diseases are dependent on larger droplets that measure in the region of 5 micrometers.

The aerosol can be generated at the test site by a variety of techniques, including simple boiling (suitable for naked oligonucleotides or natural nucleic acids—lipid and protein denature or disintegrate at 100° C. but might still encase the oligonucleotide and natural nucleic acids in a virus-like particle), ultrasonic mist, piezoelectric crystals (ink-jet printer), simple mechanical agitation, or bubbling air through a tank at a predetermined depth (pressure). Aerosol is allowed to disperse under the simulated conditions.

If the oligonucleotide tracer or natural nucleic acid is recovered (detectable in the laboratory) from a distant location, it will constitute proof that there is cross-contamination of the remote location by air-borne oligonucleotide or natural nucleic acid released at the proximate site. If ventilation work or other barriers had been enacted or in place to prevent such an occurrence before the above test, such measures can be deemed inadequate should an actual air-borne infectious disease break out.

Recovery of Oligonucleotide Tracers

The recovery of tracer oligonucleotide from a location distant from the source of aerosol means that in the event of airborne infectious disease outbreak, a patient at the location harboring that disease will present a real danger to an uninfected non-immune person at the remote location.

Oligonucleotide or natural nucleic acid tracer molecules are recovered by one of several ways, singly or in combination:
1. Swabbing—a cotton tipped sterile swab moistened with electrolyte solution. The cotton tip does not contain inhibitors to PCR, or enzymes that degrade the tracer. The electrolyte solution provides cations that bind to the negatively charged oligonucleotide molecule, facilitating the transferal from the environmental object to the swab.
2. Prior installation of “landing pad” for the oligonucleotide or natural nucleic acid tracer in the environment and removal of the pads after release of tracer for laboratory identification of any attached oligonucleotide or natural nucleic acid tracer. The pad can be plastic cling-wrap, solidified agarose gel, nylon membrane, nitrocellulose membrane, an opened test tube with or without a buffer solution to dissolve the tracer, or other material which does not bind irreversibly with the oligonucleotide tracer or degrade it and does not inhibit subsequent PCR.
3. Sampling of air by a mechanical device that passively or actively intakes environmental air together with the tracer, if present. Once inside the device, the air is bubbled through a liquid such as water and common buffers to dissolve the oligonucleotide into the liquid. Again, no inhibitors of PCR or enzymes that degrade the tracer is allowed in the liquid. The liquid is then analyzed in the laboratory for presence of the tracer.
   Methods for Dectection of Oligonucleotide or Natural Nucleic Acid Tracer

The tracer oligonucleotide can be specifically detected by a variety of techniques, such as Watson-Crick base pairing (hybridization), sequencing (determining the sequence information), restriction fragment length polymorphism (by cutting with enzymes at specific sites and determining the polymorphic information carried by the length of the subsequent fragments), or nucleic acid amplification (polymerase chain amplification or variants). Using nucleic acid amplification, as little as one copy of the tracer can be detected, greatly elevating the sensitivity of detection of the tracer by orders of magnitude. Because as few as five adenovirus or three influenza virus particles can potentially establish an infection, this invention provides a technology to simulate viruses with an extremely high sensitivity that is required by the task.

Detection and Quantitation of Oligonucleotide or Natural Nucleic Acid Tracer

Preferably, the tracer is detected by the technique of PCR or other method of nucleic acid amplification, such as those under isothermal conditions.

Several manufacturers market Real-Time Quantitative PCR machines and kits. One of these can be used for the detection and quantification of the oligonucleotide or natural nucleic acid tracer employing our specially designed primer set.

Evaluation of the Safety of the Hospital Ward Environment

To simulate a sick person with influenza A or other infection in a hospital ward, sharing a cubicle with five other patients, aerosol of 1-5 micrometer (the full spectrum of droplets or nuclei produced) is generated at the bed to simulate production of aerosol by an unmasked patient during activities such as coughing or sneezing. Samples are then taken at several predetermined intervals later on objects at the other beds, common areas such as bathrooms, or nursing station, and analyzed in the laboratory for the presence of the oligonucleotide or natural nucleic acid tracer. To simulate a disease that is spread by droplets, only larger aerosol droplets are generated. The effectiveness of various types of masks, such as three-ply surgical masks, N95 mask or N100 mask, can be evaluated by producing the aerosol in a chamber with an opening masked by one of these masks.

This procedure is modified to assess other activities, for example nasotracheal aspiration of sputum, chest physiotherapy, nebulizer therapy for drug delivery to the respiratory tract, airway mosturization by ultrasonic mist, and artificial ventilation by a variety of machines. Aspirators or other suction devices might need an inlet valve to prevent aerosol generation when the suction is abruptly turned off. Positive pressure machines, such as ventilators and nebulizers may need other modifications in order to prevent aerosols generated from acting as vehicles for dissemination of infectious agents. Modifications can only be certified by a stringent technology defined by this invention.

Before costly structural alterations and ventilation work of existing hospital wards, it is better to test any plans by creating a mock-up ward and testing it with the oligonucleotide or natural nucleic acid racer. The mock-up ward is then modified and re-tested for adequacy of the work. If the results of retesting are satisfactory, then the engineering and building work can be considered suitable for rolling out to the actual hospital wards.

Running Multiple Tests Simultaneously

Because there are many activities that need to be tested in a given mock-up hospital ward, it is in the interest of time and effort to carefully plan and design multiple oligonucleotide and/or natural nucleic acid tracers for different activities and to test them in the mock-up ward simultaneously.

For example, one set of oligonucleotide or natural nucleic acid tracer and primers are designed to evaluate small aerosols (1 micrometer diameter or less). Another set is designed to evaluate larger droplets (5 micrometer diameter or more). A third set is designed to evaluate the adequacy of three-ply surgical mask in preventing the dissemination of larger droplets. A fourth set is designed to evaluate the sputum aspirator. Finally, four other sets are designed to evaluated all of the above when the newly designed ventilation system is activated. The advantage of this approach is realized because of the virtually unlimited number of unique oligonucleotide tracers and a very large number of natural nucleic acid tracers that can be created, as explained in the sections on HOW TO MAKE SURE THAT OLIGONUCLEOTIDE TRACER IS UNIQUE” and “NATURAL NUCLEIC ACIDS AS TRACERS”.

Portable Aerosol Generator

According to the invention, the generation of aerosol is made possible by a small portable apparatus that produces a range of aerosol droplets from a liquid solution. The droplets produced range in size from less than 1 micrometer to over 10 micrometers, similar to those produced by human coughing and sneezing.

In a preferred embodiment, the apparatus consists of a can of compressed air, driven by an ozone-layer friendly propellant, with an outlet that connects tightly to a detachable stiff plastic tubing of uniform narrow lumen of known diameter (less than 2 millimeter). The outlet (nozzle) is airtight except when a triggering mechanism is released momentarily, causing the escape of air and propellant under a pressure that can be determined from the weight of the can before use and from a special calibration curve determined in the laboratory.

To load the solution into the plastic tubing, a clean, previously unused tubing is dipped all the way into and until it touches the bottom of a conical-tipped test tube containing the solution to a pre-determined level. After two to three second to allow the fluid to enter the tubing, the top end of the tube is sealed by the gloved finger, and the tube removed. The tube is tipped back to the horizontal position and excess liquid wiped off. Momentarily tilting the end that was inserted into the solution to 45 degrees above the horizontal level, and one to several gentle taps to the other end of the tube with the sealing finger, a small amount of air is permitted to escape from that end of the tube and the solution allowed to slip a little into the main body of the tube. This prevents the solution from dripping out of the tubing when it is being attached to the nozzle of the can.

Alternatively, the solution can be loaded with the help of a hypodermic syringe and the insertion of the needle into the lumen of the tube before emptying the contents in the syringe by gently pushing the piston. As a substitute, a multiple dose small dispenser with a narrow tip that easily slips into the lumen of the stiff plastic adaptor tubing can be employed.

Precautions mentioned above thus taken, the end of the adaptor tube previously sealed by the gloved finger is carefully attached firmly to the nozzle of the can. The portable aerosolizer is now ready to fire.

Because the human nose points downward, to simulate sneezing in a seated position, the tip of the tubing is pointed downward almost vertically from a height of about 1 meter from the chair or bed on which the patient is supposed to be. To simulate coughing, the tube is pointed forward, horizontal to the floor or upwards towards the ceiling. It can also be pointed sideways at bed level to simulate coughing when a person is lying in bed on the side.

To release aerosol, a quick firm squeeze is delivered to the trigger and held for one second. This effectively aerosolizes the droplet of solution inside the tubing. The rapid escape of the pressurized air further simulates the act of coughing and sneezing, which is forceful, although dependent on the strength of the individual.

Safe operation of the aerosolizer dictates that the tip of the tubing be pointed away from the operator, who takes additional precautions not to inhale excessive amount of the propellant and to evacuate the room of other occupants temporarily while the aerosol is being produced. If the room is too small and the ventilation poor, the use of excessive propellant may be not permitted. Compressed air without propellant is not limited by these considerations and is therefore preferred.

In order to better simulate spells of sneezing and coughing, which is the normal situation rather than one isolated sneeze or cough, the process can be repeated as outlined above several times. Loading the tubing with solution is relatively expeditious, especially with a little practice or with a multiple dose dispenser.

By standardizing the pressure generated (obtained from weight of can before use and calibration chart), the duration of release of air, the luminal diameter of the tubing, the depth to which it is dipped into the solution (or volume of solution dispensed into adaptor tubing), and other parameters, a standardized and fairly representative spectrum of aerosol droplets and ejection velocity can be achieved. Importantly, a range of aerosol droplets that mimics human sneezing and coughing is generated in pulses and ejection velocity that mimic those activities.

The different directions that the tip can be pointed to also enables simulation of various positions of the patient, permitting assessment of different positions of the patient and the impact on adjacent patients in a shared ward room.

In another embodiment of the portable aerosol-generating apparatus, the compressed air is supplied by manually pumping air into a small chamber, which stores the pressurized air. A pressure gauge connected to the compressed air chamber provides air pressure reading to the operator, who stops pumping when the predetermined air pressure is reached. A release mechanism operated by pulling a trigger discharges the compressed air suddenly through a narrow outlet. By connecting this outlet with an adaptor tube containing the tracer solution, the solution is aerosolized into the environment to simulate human coughing or sneezing. The air that this apparatus takes in is supplied by a bag of uncompressed atmospheric air isolated from the environment being tested, so that no recycling of air into the device occurs. In this way, the tracer does not contaminate the inside of the apparatus, so that the same apparatus can be used with other tracers. This embodiment has other advantages including being able to gauge and standardize the air pressure used to aerosolize the tracer solution and the ability to vary the air pressure to suit different simulations. No chemical propellant is needed.

Portable Air Sampler

According to the invention, an apparatus consisting of a previously empty or vacuumed, airtight, cylindrical hollow chamber, an intake port, an optional output port, an automatic or manual means to draw air, and a means to close or seal the intake port, permits the quick intake of a sample of ambient air and the transport to the laboratory for subsequent analysis. Programmable, automatic, repetitious, multiple air samplers controlled by small machines greatly facilitate the process.

In a preferred embodiment, the apparatus may be transparent or opaque, made of non-pervious (to air) glass or plastic material, is hollow, and contains a predetermined amount of buffered electrolyte solution free of RNAse and DNase. The hollow chamber takes the shape of a cylinder, one end of which is a relatively narrow opening that is closed by a rubber stopper or can be readily sealed by Parafilm M®, dough, molten wax, or other material. The other end is wide and admits a piston, which is designed to travel a given length of the chamber, so that before use, air is completely expelled, save the electrolyte fluid, and when drawn to a predetermined location or stop, takes in a sample of ambient air into the chamber, with a final air pressure identical to the outside.

In another preferred embodiment, the chamber with electrolyte fluid is vacuumed by withdrawing the air above the liquid. In this embodiment, the chamber has only one end and therefore resembles a test tube with only one end. A rubber stopper seals the end of the chamber securely. At the site where air is to be sampled, the rubber stopper is punctured with a hollow needle. The vacuum is momentarily broken and air enters the chamber by atmospheric pressure. After a brief interval, when air pressure equalizes between the inside of the chamber and the exterior, the needle is withdraw and the rubber seals the inside of the chamber from further exchange with the exterior. Alternatively, the tube may be sealed by a screw on top, which can be unscrewed momentarily to admit air into the bottle at the site of air sampling.

In both embodiments, the air that is sampled is taken back to the laboratory for further processing. For determination of the presence and quantity of tracer macromolecules previously released into the air, this invention provides a means of sampling a fixed volume of air in a room of known size, thereby giving the concentration in ppt (parts per trillion), or ppq (parts per quadrillion) of the air being sampled. In addition, this method of air sampling is portable, fast, and provides an instant snapshot of the concentration of tracer macromolecules.

The buffered electrolyte solution is particularly suitable for the extraction of electrically charged macromolecules, such as single stranded DNA, RNA, chimeric/hybrid/photo- or chemically altered DNA or RNA, double stranded DNA, DNA/RNA hybrids, DNA or RNA complexes with protein or other molecules, or microorganisms suspended in the air.

### EXAMPLES

1. Examples of the Oligonucleotides and Natural DNA and the Corresponding Primers

(1) The Sequences of a 60-mer Oliginucleotide and its Primers

| | |
|---|---|
| CATGAATTCC GCTGCATACC AATGTGTAAG | 30 |
| CTAGATCAGT CAGGACTGAT CGAATTCCAG | 60 |
| Forward primer: CATGAATTCC GCTGCATACC | 20 |
| Reverse primer: CTGGAATTCG ATCAGTCCTG | 20 |

(2) The Sequences of a 100-mer Oligonucleotide and Primers:

| | |
|---|---|
| GGCTACCGCT CGCATACTAA TGGTGTACGA | 30 |
| CAGCTCGAGC TATCTGCGTA CGTATCGTAT | 60 |
| GCTAGCCGCG CAGTAGGAGC ACTATATCGA | 90 |
| GCTATCCATC | 100 |
| Forward primer: CGCTCGCATA CTAATGGTGT A | 21 |
| Reverse primer: TATAGTGCTC CTACTGCGCG G | 21 |

(3) The Sequences of a 100-mer Oligonucleotide and its Primers:

| | |
|---|---|
| GGCTAGGGGA TAGCTACTAG CCGTTTATTG | 30 |
| CGCTATCTAC GTACTGATCC GTAGACGCGT | 60 |
| ACATCAGCTA GCGTGCTAGC TACATCGACG | 90 |
| CGCATTACGA | 100 |
| Forward primer: TACTAGCCGT TTATTGCGCT | 20 |
| Reverse primer: TAATGCGCGT CGATGTAGCT | 20 |

(4) The Sequences of a 100-mer Oligonucleotide and its Primers:

| | |
|---|---|
| CCGCTAGCGT ATATCGCGCC GATATCTACG | 30 |
| CGGCTATACT ACCATTACGA CGATGCGCGC | 60 |
| TTATCACACG GAGCATCGAC GTACGATTCG | 90 |
| GTATTATTCA | 100 |
| Forward primer: TATATCGCGC CGATATCTAC G | 11 |
| Reverse primer: ATACCGAATC GTACGTCGAT G | 11 |

(5) Sequence of a Segment of the Human Beta-Actin cDNA (B1) and Primers

Sequence of Tracer B1:

| | |
|---|---|
| GATATCGCCG CGCTCGTCGT CGACAACGGC | 30 |
| TCCGGCATGT GCAAGGCCGG CTTCGCGGGC | 60 |
| GACGATGCCC CCCGGGCCGT CTTCCCCTCC | 90 |
| ATCGTGGGGC GCCCCAGGCA CCAGGGCGTG | 120 |
| ATGGTGGGCA TGGGTCAGAA GGATTCCTAT | 150 |
| GTGGGCGACG AGGCCCAGAG CAAGAGAGGC | 180 |
| ATCCTCACCC TGAAGTACCC CATCGAGCAC | 210 |
| GGCATCGTCA CCAACTGGGA CGACATGGAG | 240 |
| AAAATCTGGC ACCACACCTT CTACAATGAG | 270 |
| CTGCGTGTGG CTCCCGAGGA GCACCCCGTG | 300 |
| CTGCTGACCG AGGCCCCCCT GAACCCCAAG | 330 |
| GCCAACCGCG AGAAGATGAC CCAGATCATG | 360 |
| TTTGAGACCT TCAACACCCC AGCCATGTAC | 390 |
| GTTGCTATCC AGGCTGTGCT ATCCCTGTAC | 420 |
| GCCTCTGGCC GTACCACTGG CATCGTGATG | 450 |
| GACTCCGGTG ACGGGGTCAC CCACACTGTG | 480 |
| CCCATCTACG AGGGGTATGC CCTCCCCCAT | 510 |
| GCCATCCTGC GTCTGGACCT GGCTGGCCGG | 540 |
| GACCTGACTG ACTACCTCAT GAAGATCCTC | 570 |
| ACCGAGCGCG GCTACAGCTT CACCACCACG | 600 |
| GCCGAGCGGG AAATCGTGCG TGACATTAAG | 630 |
| GAGAAGCTGT GCTACGTCGC CCTGGACTTC | 660 |
| GAGCAAGAGA TGGCCACGGC TGCTTCCAGC | 690 |
| TCCTC | 695 |

Primers Used for Detection of Tracer B1:

| | | |
|---|---|---|
| B1-F (OL322): | 5′-GACTACCTCA TGAAGATCCT C | 21 |
| B1-R1 (OL323): | 5′-CGTGGCCATC TCTTGCTCG | 19 |
| B1-R2 (OL324): | 5′-AAGTCCAGGG CGACGTAGC | 19 |

B1-F and B1-R1 are used for the 1^{st} round PCR with 129 bp PCR products.
B1-F and B1-R2 are used for the 2^{nd} round PCR with 110 bp PCR products.
(6) Sequence of a Segment of the Human GAPDH cDNA (G2) and Primers
Sequence of Tracer G2:

The underlined parts are the corresponding sites of primers for preparation of Tracer G2 by PCR.

| | |
|---|---|
| CCCATCACCA TCTTCCAGGA GCGAGATCCC | 30 |
| TCCAAAATCA AGTGGGGCGA TGCTGGCGCT | 60 |
| GAGTACGTCG TGGAGTCCAC TGGCGTCTTC | 90 |
| ACCACCATGG AGAAGGCTGG GGCTCATTTG | 120 |
| CAGGGGGGAG CCAAAAGGGT CATCATCTCT | 150 |
| GCCCCCTCTG CTGATGCCCC CATGTTCGTC | 180 |
| ATGGGTGTGA ACCATGAGAA GTATGACAAC | 210 |
| AGCCTCAAGA TCATCAGCAA TGCCTCCTGC | 240 |
| ACCACCAACT GCTTAGCACC CCTGGCCAAG | 270 |
| GTCATCCATG ACAACTTTGG TATCGTGGAA | 300 |
| GGACTCATGA CCACAGTCCA TGCCATCACT | 330 |
| GCCACCCAGA AGACTGTGGA TGGCCCCTCC | 360 |
| GGGAAACTGT GGCGTGATGG CCGCGGGGCT | 390 |
| CTCCAGAACA TCATCCCTGC CTCTACTGGC | 420 |
| GCTGCCAAGG CTGTGGGCAA GGTCATCCCT | 450 |
| GAGCTGAACG GGAAGCTCAC TGGCATGGCC | 480 |
| TTCCGTGTCC CCACTGCCAA CGTGTCAGTG | 510 |
| GTGGACCTGA CC | 522 |

Primers Used for Detection of Tracer G2

| | | |
|---|---|---|
| G2-F (OL37): | 5′-CCCATCACCA TCTTCCAGG | 19 |
| G2-R1 (OL325): | 5′-GATCTTGAGG CTGTTGTCAT AC | 22 |
| G2-R2 (OL326): | 5′-GAGCCCCAGC CTTCTCCATG | 20 |

G2-F and G2-R1 are used for the 1^{st} round PCR with 222 bp PCR products.
G2-F and G2-R2 are used for the 2^{nd} round PCR with 115 bp PCR products.
2. Assessment of a Hospital Ward for Adequacy of Ventilation

This example illustrates how to apply this invention in the assessment of the safety of hospital wards in the prevention of nosocomial transmission of air-borne infectious diseases.

FIG. 1 depicts a six-bed hospital ward cubicle (1). The beds (2 and 3) are labeled alphabetically (A-F). Curtains or partitions (4) and individual ventilation exhausts (5) are installed for privacy and/or barriers against the spread of air-borne disease. In this example, bed A is assumed to have received an index patient (2) suffering from an air-borne infectious disease. The other patients (3) are not previously infected by or immune to that disease and are therefore susceptible to nosocomial infection. Because of the presence of the index patient (2) in the same cubicle, they are now at risk of being infected if the barriers and exhaust are inadequate. Assuming that the index patient does not venture over to the other beds and that medical workers strictly observe hand-washing between patient care and other guidelines, the air becomes the only medium for transmission of the infectious agent.

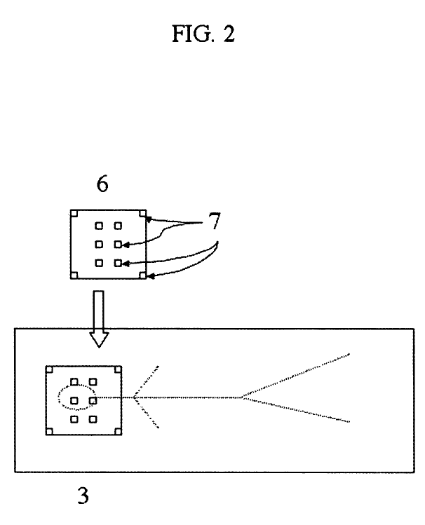

FIG. 2 shows how to prepare for testing using this invention. In each of the other beds are laid a previously fabricated square cardboard (6) measuring 1.0×1.0 meter. Pasted on the cardboard are ten “landing pads” (7) to receive any tracer that happens to landing on them. These “landing pads” (7) measure 1 cm×1 cm each. Together, they represent 10⁻⁴ of the area of the cardboard, thus permitting an estimation of the amount of tracer that deposits on the remote location (another bed). The “landing pads” (7) are subsequently peeled off and inserted into test tubes for transportation back to the laboratory.

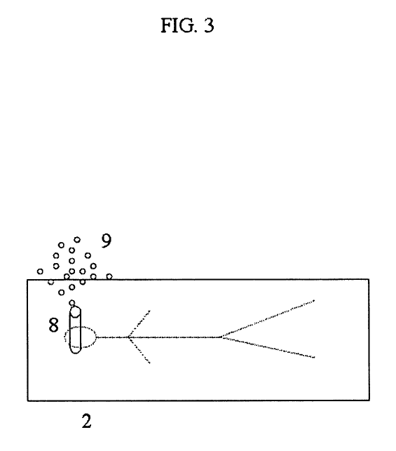

FIG. 3 illustrates the simulation of the index patient (2) by machine (8) generation of aerosol (9) ejected at speeds that simulate breathing, coughing or sneezing. With separate simulation runs using one oligonucleotide or natural nucleic acid tracer with the exhaust (5) off and another oligonucleotide or natural nucleic acid tracer with the exhaust turned on, the efficacy of the exhaust in prevention of air-borne contamination of another bed can be evaluated.

Such evaluations is necessary because a patient shedding infectious viruses in the respiratory secretions may not be identified as harboring the disease agent (silent carrier) and may therefore be sharing a body of air with other susceptible individuals (patients, visitors, and health care professionals).

The sampling of an exact volume of air under atmospheric pressure enables the determination of quantitative information. By repeatedly sampling the air at a given location, multiple snap-shots of the situation can be taken to reconstruct the temporal features of the contamination (similar to shooting a movie). Sampling different areas in the hospital ward provide information on the spatial dispersion of the simulated air-borne infectious disease agent, and may lead to correction of deficiencies in the ventilation, partitioning of common spaces, trafficking of people, use of exhaust fans, distance between and orientation of hospital beds, use of medical equipments, such as supplemental oxygen, nebulizer, or suctioning and other situations.

Design of new hospitals and expensive modifications to existing hospital wards can proceed after a model ward is thoroughly tested and certified by this invention.

3. Certification of the Safety of a Microbiology Laboratory or Biological Safety Cabinet

The microbiology laboratory or biological safety cabinet is a small version of an infectious disease ward or room.

Infectious disease agents are confined to the interior of the restricted microbiology laboratory and the biological safety cabinet, filtered and trapped from the exhaust air, and exterminated periodically, so that none escapes to the exterior.

Using techniques similar to those described in Example 2, these facilities can be tested for safety and certified periodically, to ensure safety to laboratory workers and the community.

4. Modelling the Outbreak of SARS in Amoy Gardens in Hong Kong

In March, 2003, an outbreak of SARS occurred in Amoy Gardens, Hong Kong. Investigators in the University of Hong Kong quickly pinpointed the possible culprit—faulty drainage system that acted as gigantic aerosol amplifiers, with the result of dissemination of infectious aerosol to other building blocks by air.

Although the hypothesis was shown to be plausible by computer simulations, proof of the hypothesis was not possible because of the lack of a sensitive and specific technology, a technology that permits multiple simultaneous experiments, and one that is safe to the environment and occupants.

Dispersion analysis by other tracers are limited to short distances because technology to detect the tracers does not work with longer distances because of dilution of the tracers.

These tracers are also limited in variety and permits only serial experiments. For example, while the drainage system was shown to be faulty, other hypothesis have been advanced to explain the aerosol, namely forceful flushing of the toilet, aerosol generated by hot shower and the production of aerosol during defecation of infected stool. To simulate all of these activities, it would be desirable to have a technology that permits simultaneous experiments to be carried out.

While smoke or other tracers are relatively safe when used in small amounts, the large amounts that need to be used to cover the long distance of dispersion seen in the Amoy Garden outbreak may cause concern. Also, if hot smoke is used, it has a tendency to ascend, rather than to faithfully replicate the ambient conditions during simulation.

Gases have the property to fill up all the space in a container. Using gaseous tracers in an enclosed environment such as a hospital ward, therefore suffers from the drawback that it does not behave like individual virus particles or droplets of moisture containing viral particles. Individual virus particles and droplets of moisture ejected into the air are carried around by convection or other currents instead of behaving as gases and will drop under the influence of gravity if the air is undisturbed.

This invention provides oligonucleotides and natural nucleic acids that do not suffer from the above-mentioned disadvantages of other tracers because when suspended in the air, they move around passively like virus particles or droplets of moisture but are completely safe to the environment and living things. In addition, because our tracers can be amplified by PCR, the sensitivity of detection is markedly improved over other tracers. The oligonucleotide or natural nucleic acid tracer is also stable and therefore can cover vast distances. Moreover, multiple oligonucleotide or natural nucleic acid tracers can be used to simulate the various hypothetical conditions at the same time, giving not only faster results but also costing much less and causing least inconvenience to the occupants.

This strategy of modeling a disease outbreak is not limited to airborne diseases. It can be used to simulate diseases spread by oral-fecal route or by fomites (touch).

This invention can also be used to study contamination of surface water systems and underground water by industrial pollutants. Cities complaining of air pollution from upwind cities might also find this method useful to make a case.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

### REFERENCES

1. Papineni R S, Rosenthal F S. The size distribution of droplets in the exhaled breath of healthy human subjects. J Aerosol Med. 1997 Summer;10(2):105-16.
1. A New Hypothesis for the SARS Outbreak in Amoy Gardens Block E (http://www.hku.hk/uhs/he/flu/press releases/pr-20030429-01-e.pdf).
2. Musher D M. How contagious are common respiratory tract infections? N Engl J Med. 2003 Mar. 27;348(13): 1256-66.
3. Self-Hagensee M E, Yaegashi N, Galloway D A. of human papillomavirus type 1 capsids by expression of the L1 protein alone or by coexpression of the L1 and L2 capsid proteins. J Virol. 1993 January;67(1):315-22.
4. American Standard Code for Information Interchange at http://www.encyclopedia.com/html/A/AS1C1|1|1.asp Nicholson G P, Clark R P, dE Calcina-Goff M L. “Theoretical and practical compari
5. son for the potassium iodide tracer method (KI-Discus) for assessing the containment efficiency of fume cupboards with the gas tracer method described in BS 7258: 1994: Part 4”, Annals of Occupational Hygiene, Vol. 43, No. 4, 1999, 257-267.
6. Hitchings D T. “Laboratory fume hood and exhaust fan penthouse exposure risk analysis using the ANSI/ASHRAE 110-1995 and other tracer gas methods”, ASHRAE Transactions, Vol. 103, Pt 2, 1997, 863-872.
7. Wong C Y C. Severe acute respiratory syndrome and biology, air quality, physics, and mechanical engineering. HK Med J. 2003. 9(4):304-305.

1. A method of determining whether a facility can effectively prevent the spread of an infectious disease comprising:
a) releasing an effective amount of biological tracers in or near the facility, to mimic the spread of pathogens that cause the infectious disease; and
b) detecting the presence or amount of the biological tracers to determine the spread of the biological tracers in the environment of the facility.

2. The method of claim 1 wherein the facility is used to prevent the spread of the infectious disease from the outside into the facility, the biological tracers are released outside the facility, and the presence or amount of the biological tracers is detected in the facility.

3. The method of claim 1 wherein the facility is used to prevent the spread of the infectious disease from the inside to the outside of the facility, the biological tracers are released in the facility, and the presence or amount of the biological tracers is detected outside the facility.

4. The method of claim 1 wherein the biological tracers are unique from any other biological molecules existing in the facility and its environment.

5. The method of claim 1 further comprising collecting a sample in or near the facility, the presence or amount of the biological tracers is determined by analyzing the sample.

6. The method of claim 5 wherein the sample is collected from the medium in or near the facility, wherein the medium is selected from the group consisting of gas, liquid and solid substance.

7. The method of claim 6 wherein the gas substance is air, to thereby determine whether the infectious disease has spread into the air in or near the facility.

8. The method of claim 6 wherein the liquid is water, to thereby determine whether the infectious disease has spread into the water in or near the facility.

9. The method of claim 6 wherein the solid substance is collected from the surface of a solid object, to thereby determine whether the biological tracer has landed on or adhered to the surface of the solid object.

10. The method of claim 1 wherein the biological tracers are nucleic acids.

11. The method of claim 10 wherein the nucleic acids are selected from the group consisting of single stranded DNAs, single stranded RNAs, double stranded DNAs, double stranded RNAs, DNA/RNA hybrids, chimeric DNA molecules, chimeric RNA molecules, plasmid DNAs, and DNAs and RNA extracted from an organism.

12. The method of claim 11 wherein the organism is selected from the group consisting of E. coli, yeast, and other organisms.

13. The method of claim 10 wherein the nucleic acids are identified by a method comprising a step selected from the group consisting of nucleic acid hybridization, polymerase chain reaction, restriction fragment length polymorphism, and sequence information.

14. The method of claim 10 wherein each of the nucleic acids consists of any number of nucleotides.

15. The method of claim 10 wherein each of the nucleic acids consists of between 100-5,000 nucleotides.

16. The method of claim 10 wherein each of the nucleic acids consists of between 40-100 nucleotides.

17. The method of claim 1 wherein the detecting step comprising amplifying the biological tracers.

18. The method of claim 17 wherein nucleic acid amplification technology is used to amplify the biological tracers.

19. The method of claim 1 wherein the facility is selected from the group consisting of ventilation system and drainage system.

20. The method of claim 1 wherein the biological tracer is provided in a form selected from the group consisting of aerosol and solution.

21. The method of claim 20 wherein the solution of the biological tracer comprises an additive substance that increases the viscosity of the solution to more closely mimic the body fluids in which viruses are released into the environment.

22. The method of claim 21 wherein the substance that increases the viscosity of the solution is selected from the group consisting of proteins, gelatin, and glycerol.

23. The method of claim 20 wherein the infectious disease is an airborne disease, the solution is released in the facility in a range of fairly uniformly sized droplets, and the droplets have a diameter of equal to or less than 1 micrometer.

24. The method of claim 20 wherein the infectious disease is droplet-disseminated disease, the solution is released in the facility in a range of substantially uniformly sized droplets, and the droplets have a diameter of from 1 to 10 micrometer.

25. The method of claim 20 wherein the concentration of the solution ranges from 2×10⁻¹⁵ to 1 mmole/l, preferably from 2×10⁻¹⁵ to 2×10⁻⁵ mmole/l, and even more preferably from 2×10⁻¹⁵ to 2×10⁻⁹ mmole/l.

26. The method of claim 20 wherein the concentration of the solution ranges from 10³ to 5×10¹⁷ copies/ml, preferably from 10³ to 10¹³ copies/ml, and even more preferably from 10³ to 10⁹ copies/ml.

27. The method of claim 1 wherein the biological tracer is not encapsulated by a polymer.

28. The method of claim 1 wherein the biological tracer is encapsulated by a polymer.

29. The method of claim 28 wherein the polymer is selected from the group consisting of protein molecules, lipid molecules, carbohydrates, and combination thereof.

30. The method of claim 1 wherein the sequences of the biological tracers encode information selected from the group consisting of manufacturer, manufacture date, sequence identification, lot number, and combinations thereof.

31. The method of claim 1 wherein the detection of the biological tracers at different locations at a given time after release of said tracers within the facility provides spatial information on the dispersal of said tracers.

32. The method of claim 1 wherein the detection of the biological tracers at different times after the release of said tracers at the same spot within the facility provides temporal information on the dispersal of said tracers.

33. The method of claim 1 wherein the detection of the biological tracers at different times and at different locations after the release of said tracers within the facility provides temporal and spatial information on the dispersal of said tracers.

34. An apparatus for generating aerosol comprising a chamber of compressed gas having a nozzle that is attached to a detachable stiff tubing capable of holding a solution of predetermined volume.

35. The apparatus of claim 34 further comprising a trigger to open the nozzle so as to discharge the compressed gas through the tubing.

36. The apparatus of claim 34 wherein the chamber can be manually filled with compressed air, the pressure of which can be monitored by a pressure gauge.

37. The apparatus of claim 34 wherein the stiff tubing has a uniform internal luminal diameter.

38. A method of generating aerosol that mimic the results of a human sneeze or cough comprising passing compressed air in pulses through a narrow tube that holds a solution being aerosolized.

39. The method of claim 38 wherein the repeated generation of pulses of aerosol simulate spells of coughing or sneezing.

40. An apparatus that samples a body of air by suctioning into a vacuumed container through a temporary puncture or by manually drawing a predetermined volume of air into a chamber using a piston.

41. The apparatus of claim 40 wherein the vacuumed container or the chamber is sealed before being brought into the body of air being sampled.

42. The apparatus of claim 40 wherein the vacuumed container or the chamber contains a DNase-free electrolyte solution.

43. The apparatus of claim 40 wherein the container or the chamber is sealed after a sample of air is taken into the container or chamber.


SEQUENCE LISTING (20 sequences)

SEQ ID NO: 1 - DNA, 60 bp, Artificial Sequence
  Description of Artificial Sequence Synthetic oligonucleotide
catgaattccgctgcataccaatgtgtaagctagatcagtcaggactgatcgaattccag

SEQ ID NO: 2 - DNA, 20 bp, Artificial Sequence
  Description of Artificial Sequence Primer
catgaattccgctgcatacc

SEQ ID NO: 3 - DNA, 20 bp, Artificial Sequence
  Description of Artificial Sequence Primer
ctggaattcgatcagtcctg

SEQ ID NO: 4 - DNA, 100 bp, Artificial Sequence
  Description of Artificial Sequence Synthetic oligonucleotide
ggctaccgctcgcatactaatggtgtacgacagctcgagctatctgcgtacgtatcgtatgctagccgcgcagtaggagcactatatcgagctatccatc

SEQ ID NO: 5 - DNA, 21 bp, Artificial Sequence
  Description of Artificial Sequence Primer
cgctcgcatactaatggtgta

SEQ ID NO: 6 - DNA, 21 bp, Artificial Sequence
  Description of Artificial Sequence Primer
tatagtgctcctactgcgcgg

SEQ ID NO: 7 - DNA, 100 bp, Artificial Sequence
  Description of Artificial Sequence Synthetic oligonucleotide
ggctaggggatagctactagccgtttattgcgctatctacgtactgatccgtagacgcgtacatcagctagcgtgctagctacatcgacgcgcattacga

SEQ ID NO: 8 - DNA, 20 bp, Artificial Sequence
  Description of Artificial Sequence Primer
tactagccgtttattgcgct

SEQ ID NO: 9 - DNA, 20 bp, Artificial Sequence
  Description of Artificial Sequence Primer
taatgcgcgtcgatgtagct

SEQ ID NO: 10 - DNA, 100 bp, Artificial Sequence
  Description of Artificial Sequence Synthetic oligonucleotide
ccgctagcgtatatcgcgccgatatctacgcggctatactaccattacgacgatgcgcgcttatcacacggagcatcgacgtacgattcggtattattca

SEQ ID NO: 11 - DNA, 21 bp, Artificial Sequence
  Description of Artificial Sequence Primer
tatatcgcgccgatatctacg

SEQ ID NO: 12 - DNA, 21 bp, Artificial Sequence
  Description of Artificial Sequence Primer
ataccgaatcgtacgtcgatg

SEQ ID NO: 13 - DNA, 695 bp, Homo sapiens
gatatcgccgcgctcgtcgtcgacaacggctccggcatgtgcaaggccggcttcgcgggcgacgatgccccccgggccgtcttcccctccatcgtggggcgccccaggcaccagggcgtgatggtgggcatgggtcagaaggattcctatgtgggcgacgaggcccagagcaagagaggcatcctcaccctgaagtaccccatcgagcacggcatcgtcaccaactgggacgacatggagaaaatctggcaccacaccttctacaatgagctgcgtgtggctcccgaggagcaccccgtgctgctgaccgaggcccccctgaaccccaaggccaaccgcgagaagatgacccagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctc

SEQ ID NO: 14 - DNA, 21 bp, Artificial Sequence
  Description of Artificial Sequence Primer
gactacctcatgaagatcctc

SEQ ID NO: 15 - DNA, 19 bp, Artificial Sequence
  Description of Artificial Sequence Primer
cgtggccatctcttgctcg

SEQ ID NO: 16 - DNA, 19 bp, Artificial Sequence
  Description of Artificial Sequence Primer
aagtccagggcgacgtagc

SEQ ID NO: 17 - DNA, 522 bp, Homo sapiens
cccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacc

SEQ ID NO: 18 - DNA, 19 bp, Artificial Sequence
  Description of Artificial Sequence Primer
cccatcaccatcttccagg

SEQ ID NO: 19 - DNA, 22 bp, Artificial Sequence
  Description of Artificial Sequence Primer
gatcttgaggctgttgtcatac

SEQ ID NO: 20 - DNA, 20 bp, Artificial Sequence
  Description of Artificial Sequence Primer
gagccccagccttctccatg